(19) United States
(12) Patent Application Publication
(10) Pub. No.: US 2022/0351830 A1
(43) Pub. Date: Nov. 3, 2022

(54) SYSTEM AND METHOD FOR GENERATING A HABIT DYSFUNCTION NOURISHMENT PROGRAM

(71) Applicant: KPN INNOVATIONS, LLC., Lakewood, CO (US)

(72) Inventor: Kenneth Neumann, LAKEWOOD, CO (US)

(73) Assignee: KPN INNOVATIONS, LLC., LAKEWOOD, CO (US)

(21) Appl. No.: 17/243,662

(22) Filed: Apr. 29, 2021

(51) Int. Cl. G16H 20/60; G06N 20/00

(52) U.S. Cl. CPC G16H 20/60; G06N 20/00

(57) ABSTRACT

A system for generating a habit dysfunction nourishment program includes a computing device configured to obtain a habit indicator, identify a habit profile, wherein identifying the habit profile further comprises, retrieving a behavioral parameter, determining a behavioral divergence as a function of the behavioral parameter, and identifying the habit profile as a function of the behavioral divergence and the habit indicator using a habit machine-learning model, determine an edible as a function of the habit profile, and generate a nourishment program as a function of the edible.

### FIELD OF THE INVENTION

The present invention generally relates to the field of artificial intelligence. In particular, the present invention is directed to a system and method for generating a habit dysfunction nourishment program.

### BACKGROUND

Current edible suggestion systems do not account for the lifestyle of an individual. This leads to inefficiency of an edible suggestion system and a poor nutrition plan for the individual. This is further complicated by a lack of uniformity of nutritional plans, which results in dissatisfaction of individuals.

### SUMMARY OF THE DISCLOSURE

In an aspect, a system for generating a habit dysfunction nourishment program includes a computing device configured to obtain a habit indicator, identify a habit profile, wherein identifying the habit profile further comprises, retrieving a behavioral parameter, determining a behavioral divergence as a function of the behavioral parameter, and identifying the habit profile as a function of the behavioral divergence and the habit indicator using a habit machine-learning model, determine an edible as a function of the habit profile, and generate a nourishment program as a function of the edible.

In another aspect, a method for generating a habit dysfunction nourishment program includes obtaining, by a computing device, a habit indicator, identifying, by the computing device, a habit profile, wherein identifying the habit profile further comprises, retrieving a behavioral parameter, determining a behavioral divergence as a function of the behavioral parameter, and identifying the habit profile as a function of the behavioral divergence and the habit indicator using a habit machine-learning model, determining, by the computing device, an edible as a function of the habit profile, and generating, by the computing device, a nourishment program as a function of the edible.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating an exemplary embodiment of a system for generating a habit dysfunction nourishment program;

FIG. 2 is a block diagram illustrating an exemplary embodiment of a behavioral parameter;

FIG. 3 is a block diagram illustrating an exemplary embodiment of an edible directory;

FIG. 4 is a block diagram illustrating an exemplary embodiment of a corporeal effect;

FIG. 5 is a block diagram of an exemplary embodiment of a machine-learning module;

FIG. 6 is a process flow diagram illustrating an exemplary embodiment of a method for generating a habit dysfunction nourishment program; and

FIG. 7 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for generating a habit dysfunction nourishment program. In an embodiment, this disclosure can be used to obtain a habit indicator. Aspects of the present disclosure can be used to identify a habit profile. This is so, at least in part, because the disclosure incorporates a machine-learning model. Aspects of the present disclosure can also be used to determine an edible as a function of the habit profile. Aspects of the present disclosure allow for generating a nourishment program. Exemplary embodiments illustrating aspects of the present disclosure are described below in the context of several specific examples.

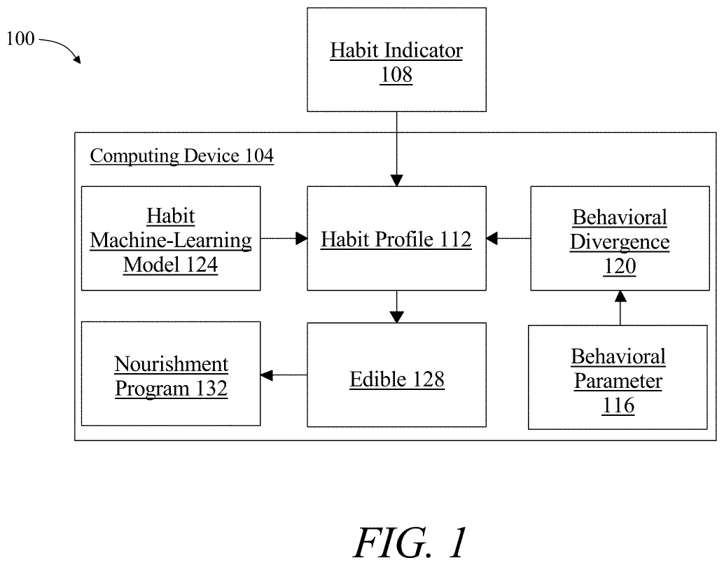

Referring now to FIG. 1, an exemplary embodiment of a system 100 for generating a habit dysfunction nourishment program is illustrated. System includes a computing device 104. Computing device 104 may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Computing device 104 may include a single computing device operating independently, or may include two or more computing device operating in concert, in parallel, sequentially or the like; two or more computing devices may be included together in a single computing device or in two or more computing devices. Computing device 104 may interface or communicate with one or more additional devices as described below in further detail via a network interface device. Network interface device may be utilized for connecting Computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device 104 may include but is not limited to, for example, a computing device or cluster of computing devices in a first location and a second computing device or cluster of computing devices in a second location. Computing device 104 may include one or more computing devices dedicated to data storage, security, distribution of traffic for load balancing, and the like. Computing device 104 may distribute one or more computing tasks as described below across a plurality of computing devices of computing device, which may operate in parallel, in series, redundantly, or in any other manner used for distribution of tasks or memory between computing devices. Computing device 104 may be implemented using a “shared nothing” architecture in which data is cached at the worker, in an embodiment, this may enable scalability of system 100 and/or computing device.

With continued reference to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, computing device 104 obtains a habit indicator 108. As used in this disclosure a “habit indicator” is an element of data representing a measurable value of an individual's biological system as a function of repeated patterns of activity, such as but not limited to an individual's habits and/or tendencies. In an embodiment, and without limitation, habit indicator 108 may denote a health status of the individual's biological system, wherein a health status is a measure of the relative level of physical, social and/or behavioral well-being. In another embodiment, habit indicator 108 may denote one or more health statuses of an individual's nervous system, circulatory system, musculoskeletal system, respiratory system, endocrine system, integumentary system, lymphatic system, digestive system, urinary system, reproductive system, and the like thereof. In an embodiment and without limitation, habit indicator 108 may include a biological sample. As used in this disclosure a “biological sample” is one or more biological specimens collected from an individual. Biological sample may include, without limitation, exhalate, blood, sputum, urine, saliva, feces, semen, and other bodily fluids, as well as tissue. Habit indicator 108 may include a biological sampling device. Habit indicator 108 may include one or more biomarkers. As used in this disclosure a “biomarker” is a molecule and/or chemical that identifies the status of an individual's health system and/or stress of an individual's health system. As a non-limiting example, biomarkers may include serum liver enzymes, c-reactive protein, GGT, ALT, cholesterol, HDL, LDL, triglycerides, glucose, serum uric acid, interleukin-6, PC SK, PPARG, UCP1, LEP, GHRL, and the like thereof.

In an embodiment, and still referring to FIG. 1, habit indicator 108 may be obtained as a function of receiving a habit input. As used in this disclosure a “habit input” is one or more inputs relating to an individual's lifestyle choices and/or habits. For example, and without limitation, habit input may include an input from one or more monitoring devices. As used in this disclosure “monitoring device” is an electronic device that is worn on the person of a user, such as without limitation close to and/or on the surface of the skin, wherein the device can detect, analyze, and transmit habit inputs to computing device 104. Monitoring device may include, without limitation, any device that further collects, stores, and analyzes data associated with habit indicator 108. Monitoring device my consist of, without limitation, near-body electronics, on-body electronics, in-body electronics, electronic textiles, smart watches, smart glasses, smart clothing, fitness trackers, body sensors, wearable cameras, head-mounted displays, body worn cameras, Bluetooth headsets, wristbands, smart garments, chest straps, sports watches, fitness monitors, and the like thereof. Monitoring device may include directed light monitoring devices such as spectrophotometric device at least identify concentrations of markers and/or identify one or more user biochemical statuses such as body mass index, fat percentage, water percentage, bone mass percentage, muscle mass percentage, and the like thereof. Monitoring device may include, without limitation, earphones, earbuds, headsets, bras, suits, jackets, trousers, shirts, pants, socks, bracelets, necklaces, brooches, rings, jewelry, AR HMDs, VR HMDs, exoskeletons, location trackers, and gesture control wearables. Monitoring device may include one or more medical devices that are operated by one or more informed advisors, an informed advisor, wherein an informed advisor may include, without limitation nutritionists, lifestyle coaches, family physicians, primary care physicians, internists, pediatricians, pathologists, neurologists, and the like thereof. As a further non-limiting example, monitoring device may include datum from one or more devices that collect, store, and/or calculate one or more lights, voltages, currents, sounds, chemicals, pressures, and the like thereof that are associated with habit indicator 108. For example, and without limitation a device may include a magnetic resonance imaging device, magnetic resonance spectroscopy device, x-ray spectroscopy device, computerized tomography device, ultrasound device, electroretinogram device, electrocardiogram device, ABER sensor, mass spectrometer, and the like thereof.

Still referring to FIG. 1, computing device 104 may obtain habit indicator 108 by receiving a medical input. As used in this disclosure a “medical input” is an element of datum that is obtained relating to an individual's lifestyle. As a non-limiting example, medical input may include an informed advisor that enters a medical assessment comprising a physical exam, neurologic exam, blood test, urine test, imaging test, cellular and/or chemical analysis, genetic test, measurement, visual examination, and the like thereof. As a further non-limiting example, medical input may include one or more questionnaires and/or surveys that identify one or more substances that an individual consumes and/or ingests, such as but not limited to, illicit drugs, over the counter drugs, nutraceuticals, vitamins, supplements, and the like thereof. As a further non-limiting example, medical input may include one or more inputs from a family member. For example, and without limitation, a brother, sister, mother, father, cousin, aunt, uncle, grandparent, child, friend, and the like thereof may enter an individual's medical records relating to lifestyle choices.

Still referring to FIG. 1, habit indicator 108 may include one or more representations of lifestyles as a function of a psychological analysis. As used in this disclosure a “psychological analysis” is an evaluation and/or estimation of the cognitive functions of an individual. For example, and without limitation, a psychological analysis may include one or more assessments of memory, behavior, motor function, emotions, and the like thereof. Psychological analysis may identify one or more feelings and/or cognitive functions of an individual such as, but not limited to, feeling nervous, on edge, restless, unsettled, stressed, surprised, creative, imaginative, daring, adventurous, high energy, low energy, angry, calm, comfortable, contentment, peace, relaxed, loveable, slow moving, fast moving, irritable, impulsive, dull, obsessing, and the like thereof. Psychological analysis may additionally or alternatively include any psychological analysis used as a psychological analysis as described in U.S. Nonprovisional application Ser. No. 17/128,120, filed on Dec. 29, 2020, and entitled “METHODS AND SYSTEMS FOR NOURISHMENT REFINEMENT USING PSYCHIATRIC MARKERS,” the entirety of which is incorporated herein by reference.

Still referring to FIG. 1, computing device 104 identifies a habit profile 112. As used in this disclosure a “habit profile” is a profile and/or estimation of an individual's health status in relation to an individual's habits and/or lifestyle choices. For example, and without limitation, habit profile 112 may denote that an individual's health status is affected and/or defined by a lifestyle choice of smoking tobacco. As a further non-limiting example, habit profile 112 may denote that an individual's health status is enhanced as a function of a habit of strenuous exercise three times a week. Computing device 104 identifies habit profile 112 as a function of retrieving a behavioral parameter 116. As used in this disclosure a “behavioral parameter” is an element of datum representing a lifestyle choice and/or habit that adjusts and/or effects an individual's health system. In an embodiment, and without limitation, behavioral parameter 116 may include one or more parameters such as social interaction parameters, physical activity parameters, alcohol consumption parameters, substance use parameters, and the like thereof. In another embodiment, and without limitation, retrieving behavioral parameter 116 may include obtaining a geolocation element. As used in this disclosure “geolocation element” is an identification of a real-world geographical location of an individual. Geolocation element may be obtained from a radar source, remote device such as a mobile phone, and/or internet connected device location. Geolocation element may include a global positioning system (GPS) of a user. Geolocation element may include geographic coordinates that may specify the latitude and longitude of a particular location where a user is located. Geolocation element may include one or more cell-tower triangulations, wherein a cell-tower triangulation identifies at least an alpha, beta, and gamma sector. Each of the sectors identify one or more distances that an individual may be from the cell-tower. One or more cell-towers may be used in the determination of the geolocation element. For example, and without limitation, a first cell-tower may identify a mobile phone located in sector beta with a distance of 8.4 miles, wherein a second cell-tower may identify the same mobile phone in sector alpha at 23.8 miles. This may be used iteratively until the exact location of the mobile phone, and/or internet connected device may be identified. Geolocation element may include one or more received signal strength indicators (RSSI), wherein a RSSI is a measurement of the power present in a received radio signal. For example, and without limitation, RSSI may include an IEEE 802.11 wireless networking device, wherein the relative received signal strength in the wireless environment is received in arbitrary units, such that a geolocation element may be identified.

In an embodiment, and still referring to FIG. 1, geolocation element may include an industrialization vector. As used in this disclosure an “industrialization vector” is a data structure that represents one or more a quantitative values and/or measures of industrialization of a geolocation. A vector may be represented as an n-tuple of values, where n is one or more values, as described in further detail below; a vector may alternatively or additionally be represented as an element of a vector space, defined as a set of mathematical objects that can be added together under an operation of addition following properties of associativity, commutativity, existence of an identity element, and existence of an inverse element for each vector, and can be multiplied by scalar values under an operation of scalar multiplication compatible with field multiplication, and that has an identity element is distributive with respect to vector addition, and is distributive with respect to field addition. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm: l=\sqrt{Σᵢ₌₀ⁿαᵢ²}, where αᵢ is attribute number i of the vector. Scaling and/or normalization may function to make vector comparison independent of absolute quantities of attributes, while preserving any dependency on similarity of attributes. For example, and without limitation, industrialization vector may denote that industrialization is occurring at a first geolocation, wherein industrialization is not occurring at a second geolocation.

Still referring to FIG. 1, computing device 104 determines a behavioral divergence 120 as a function of behavioral parameter 116. As used in this disclosure a “behavioral divergence” is a quantitative value comprising the magnitude of divergence of a behavioral parameter from a behavior normality. As used in this disclosure a “behavior normality” is a normal and/or average behavior that an individual should be exhibiting relative to a baseline behavioral pattern, wherein a “baseline behavioral pattern,” as used herein, is a pattern of behaviors identified from a cohort of similar persons. For example, and without limitation, a baseline behavioral pattern may denote that a cohort of similar persons may all exercise for 30 minutes a week. In an embodiment, baseline behavioral pattern may be determined as a function of a geographical location, demographic data, user religion, user belief, and the like thereof. Additionally or alternatively, behavioral normality may be determined as a function of a classifier to identify a user cohort for a user, wherein a classifier is described in detail above. For example, and without limitation, a classifier may determine that a cohort of similar persons may include individuals with similar beliefs, geographical locations, and/or similar hobbies and/or interests. In an embodiment, and without limitation, baseline behavioral pattern may average the one or more cohorts of persons to establish the baseline behavioral pattern. For example, and without limitation, behavior normality may denote that an individual located in a first area should be exhibiting the behavior of no illicit drug consumption. As a further non-limiting example, behavior normality may denote that an individual may drink three alcoholic beverages on average in a second location. In an embodiment, and without limitation, behavioral divergence 120 may be determined as a function of behavior normality and a divergence threshold. As used in this disclosure a “divergence threshold” is a parameter that identifies one or more variance limits of behavioral parameter from behavior normality. As a non-limiting example, divergence threshold may determine that an individual is exhibiting severe abnormal behaviors as a function of a behavioral parameter that indicates an individual that interacts with 1 individual during the time period of a month, wherein the behavior normality denotes that an individual should maintain 12 social interactions each day. In an embodiment, and without limitation divergence threshold may include a statistical element. As used in this disclosure a “statistical element” is a statistical value computed from a plurality of values in a sample and/or population. For example, and without limitation, a statistical value may be a calculated mean, mode, median, probability distribution, and the like thereof of a plurality of previous behavior normalities and/or behavioral parameters. As a further non-limiting example, a statistical value may be a calculated mean, mode, median, probability distribution, and the like thereof of a plurality of behavior normalities and/or behavioral parameters from a group of individuals. For example, and without limitation, a group of individuals may include one or more groups denoted by demographics, such as but not limited to, race, age, ethnicity, gender, marital status, income, education, employment, and the like thereof. In an embodiment, and without limitation, statistical element may include an unsigned deviation, mean signed deviation, dispersion, normalization, standard deviation, average absolute deviation, median absolute deviation, maximum absolute deviation, and the like thereof. As a non-limiting example, behavioral divergence 120 may denote that behavior parameter 116 is one standard deviation from behavior normality representing a mean concentration of interleukin-6. As a further non-limiting example, behavioral divergence 120 may denote that behavior parameter 116 of 30 minutes of exercise once a month is divergent from a behavior normality of 30 minutes of exercise every week.

Still referring to FIG. 1, computing device 104 identifies habit profile 112 as a function of behavioral divergence 120 and habit indicator 108 using a habit machine-learning model 124. As used in this disclosure “habit machine-learning model” is a machine-learning model to produce a habit profile output given behavioral divergences and habit indicators as inputs; this is in contrast to a non-machine learning software program where the commands to be executed are determined in advance by a user and written in a programming language. Habit machine-learning model 124 may include one or more habit machine-learning processes such as supervised, unsupervised, or reinforcement machine-learning processes that computing device 104 and/or a remote device may or may not use in the determination of habit profile 112. As used in this disclosure “remote device” is an external device to computing device 104. Habit machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train habit machine-learning process as a function of a habit training set. As used in this disclosure “habit training set” is a training set that correlates a behavioral divergence and/or habit indicator to a habit profile. For example, and without limitation, a behavioral divergence of 30 for a dietary habit and a habit indicator of increased triglycerides may relate to a habit profile for poor and/or inferior dietary habits. The habit training set may be received as a function of user-entered valuations of behavioral divergences, habit indicators, and/or habit profiles. Computing device 104 may receive habit training set by receiving correlations of behavioral divergences, and/or habit indicators that were previously received and/or determined during a previous iteration of determining habit profiles. The habit training set may be received by one or more remote devices that at least correlate a behavioral divergence and/or habit indicator to a habit profile. The habit training set may be received in the form of one or more user-entered correlations of a behavioral divergence and/or habit indicator to a habit profile. Additionally or alternatively, a user may include an informed advisor, wherein an informed advisor may include, without limitation nutritionists, lifestyle coaches, family physicians, primary care physicians, internists, pediatricians, pathologists, neurologists, and the like thereof.

Still referring to FIG. 1, computing device 104 may receive habit machine-learning model from a remote device that utilizes one or more habit machine learning processes, wherein a remote device is described above in detail. For example, and without limitation, a remote device may include a computing device, external device, processor, and the like thereof. Remote device may perform the habit machine-learning process using the habit training set to generate habit profile 112 and transmit the output to computing device 104. Remote device may transmit a signal, bit, datum, or parameter to computing device 104 that at least relates to habit profile 112. Additionally or alternatively, the remote device may provide an updated machine-learning model. For example, and without limitation, an updated machine-learning model may be comprised of a firmware update, a software update, a habit machine-learning process correction, and the like thereof. As a non-limiting example a software update may incorporate a new behavioral divergence that relates to a modified habit indicator. Additionally or alternatively, the updated machine learning model may be transmitted to the remote device, wherein the remote device may replace the habit machine-learning model with the updated machine-learning model and determine the habit profile as a function of the behavioral divergence using the updated machine-learning model. The updated machine-learning model may be transmitted by the remote device and received by computing device 104 as a software update, firmware update, or corrected habit machine-learning model. For example, and without limitation habit machine-learning model 120 may utilize a random forest machine-learning process, wherein the updated machine-learning model may incorporate a gradient boosting machine-learning process. Updated machine learning model may additionally or alternatively include any machine-learning model used as an updated machine learning model as described in U.S. Nonprovisional application Ser. No. 17/106,658, filed on Nov. 30, 2020, and entitled “A SYSTEM AND METHOD FOR GENERATING A DYNAMIC WEIGHTED COMBINATION,” the entirety of which is incorporated herein by reference.

Still referring to FIG. 1, computing device 104 may determine habit profile 112 as a function of a classifier. A “classifier,” as used in this disclosure is a machine-learning model, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Computing device 104 and/or another device may generate a classifier using a classification algorithm, defined as a processes whereby a computing device 104 derives a classifier from training data. Classification may be performed using, without limitation, linear classifiers such as without limitation logistic regression and/or naïve Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers.

Still referring to FIG. 1, computing device 104 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device 104 may then calculate a likelihood table by calculating probabilities of different data entries and classification labels. Computing device 104 may utilize a naïBayes equation to calculate a posterior probability for each class. A class containing the highest posterior probability is the outcome of prediction. NaïBayes classification algorithm may include a gaussian model that follows a normal distribution. NaïBayes classification algorithm may include a multinomial model that is used for discrete counts. NaïBayes classification algorithm may include a Bernoulli model that may be utilized when vectors are binary.

With continued reference to FIG. 1, computing device 104 may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 1, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least one value. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm: l=\sqrt{Σᵢ₌₀ⁿαᵢ²}, where αᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

Still referring to FIG. 1, computing device 104 may identify habit profile 112 by determining a habit dysfunction. As used in this disclosure a “habit dysfunction” is an ailment and/or collection of ailments that develop as a function of an individual's lifestyle and impact the individual's health status. As a non-limiting example habit dysfunction may include heart disease, stroke, obesity, type II diabetes, lung cancer, Alzheimer's disease, arthritis, atherosclerosis, asthma, cancer, chronic liver disease, cirrhosis, chronic obstructive pulmonary disease, colitis, irritable bowel syndrome, hypertension, metabolic syndrome, chronic kidney failure, osteoporosis, PCOD, stroke, depression, vascular dementia, and the like thereof. In an embodiment and without limitation, habit dysfunction may include a non-communicable ailments. As used in this disclosure a “non-communicable ailment” is disease that is not transmissible directly from a first individual to a second individual. For example, and without limitation, non-communicable ailment may include Parkinson's disease, autoimmune disease, stroke, heart disease, cancer, diabetes, chronic kidney disease, osteoarthritis, osteoporosis, Alzheimer's disease, cataracts, and the like thereof. Habit dysfunction may be determined as a function of one or more dysfunction machine-learning models. As used in this disclosure a “dysfunction machine-learning model” is a machine-learning model to produce a habit dysfunction output given habit indicators as inputs; this is in contrast to a non-machine learning software program where the commands to be executed are determined in advance by a user and written in a programming language. Dysfunction machine-learning model may include one or more dysfunction machine-learning processes such as supervised, unsupervised, or reinforcement machine-learning processes that computing device 104 and/or a remote device may or may not use in the determination of habit dysfunction. A dysfunction machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train dysfunction machine-learning process as a function of a dysfunction training set. As used in this disclosure “dysfunction training set” is a training set that correlates a habit indicator to a habit dysfunction. As a non-limiting example a habit indicator of an elevated concentration of triglycerides may relate to a habit dysfunction of type II diabetes. The dysfunction training set may be received as a function of user-entered valuations of habit indicators and/or habit dysfunctions. Computing device 104 may receive dysfunction training by receiving correlations of habit indicators and/or habit dysfunctions that were previously received and/or determined during a previous iteration. The dysfunction training set may be received by one or more remote devices that at least correlate habit indicators to habit dysfunctions, wherein a remote device is an external device to computing device 104, as described above. The dysfunction training set may be received by one or more user-entered correlations of habit indicators to habit dysfunctions. Additionally or alternatively, a user may include an informed advisor, wherein an informed advisor may include, without limitation nutritionists, lifestyle coaches, family physicians, primary care physicians, internists, pediatricians, pathologists, neurologists, and the like thereof.

Still referring to FIG. 1, computing device 104 may receive dysfunction machine-learning model from a remote device that utilizes one or more dysfunction machine learning processes, wherein a remote device is described above in detail. For example, and without limitation, a remote device may include a computing device, external device, processor, and the like thereof. Remote device may perform the dysfunction machine-learning process using the dysfunction training set to generate habit dysfunction and transmit the output to computing device 104. Remote device may transmit a signal, bit, datum, or parameter to computing device 104 that at least relates to habit dysfunctions. Additionally or alternatively, the remote device may provide an updated machine-learning model. For example, and without limitation, an updated machine-learning model may be comprised of a firmware update, a software update, a dysfunction machine-learning process correction, and the like thereof. As a non-limiting example a software update may incorporate a new habit indicator that relates to a modified habit dysfunction. Additionally or alternatively, the updated machine learning model may be transmitted to the remote device, wherein the remote device may replace the dysfunction machine-learning model with the updated machine-learning model and determine the habit dysfunction as a function of the habit indicator using the updated machine-learning model. The updated machine-learning model may be transmitted by the remote device and received by computing device 104 as a software update, firmware update, or corrected dysfunction machine-learning model. For example, and without limitation dysfunction machine-learning model may utilize a neural net machine-learning process, wherein the updated machine-learning model may incorporate hierarchical clustering machine-learning process. Updated machine learning model may additionally or alternatively include any machine-learning model used as an updated machine learning model as described in U.S. Nonprovisional application Ser. No. 17/106,658, the entirety of which is incorporated herein by reference. In an embodiment, and without limitation, dysfunction machine-learning model may identify habit dysfunction as a function of one or more classifiers, wherein a classifier is described above in detail.

Still referring to FIG. computing device 104 may identify habit profile 112 as a function of determining a temporal element. As used in this disclosure a “temporal element” is an element of datum representing a time period that an individual has performed a behavior and/or committed to a lifestyle, wherein a time period is described above in detail and includes measurements such as seconds, minutes, hours, days, weeks, months, years, and the like thereof. For example and without limitation, temporal element may denote that an individual has been smoking cigarettes for a time period of 20 years. As a further non-limiting example, temporal element may denote that an individual has been ingesting illicit drugs for a time period of 1 month. As a further non-limiting example, temporal element may denote that an individual has been adhering to a vegetarian diet for 2 weeks. As a further non-limiting example, temporal element may denote that an individual has been exercising for 30 minutes a day for 10 months.

Still referring to FIG. 1, computing device 104 may identify habit profile 112 as a function of determining a corporeal effect. As used in this disclosure a “corporeal effect” is an effect and/or influence that habit indicator 108 has on an individual's body. For example, and without limitation, corporeal effect may include one or more psychological symptoms including, but not limited to, becoming easily agitated, frustration, difficulty relaxing, low energy, headaches, upset stomach, tense muscles, chest pains, rapid heart rate, insomnia, nervousness, tinnitus, dry mouth, difficulty swallowing, memory loss, cardiovascular disease, obesity, sexual dysfunction, acne, psoriasis, eczema, gastrointestinal problems, and the like thereof. In an embodiment, and without limitation, corporeal effect may be an effect and/or influence that habit dysfunction has on an individual's body. For example. And without limitation, corporeal effect may include a physiological symptom such as, shortness of breath, difficulty breathing, oxidative stress, systemic inflammation, anemia, pulmonary hypertension, and the like thereof as a result of the habit dysfunction chronic obstructive pulmonary disease. Computing device 104 may identify corporeal effect as a function of receiving a target tissue, wherein a target tissue is one or more tissues and/or cells that are affected by a lifestyle choice and/or habit. For example, and without limitation, target tissue may be received as a function of one or more one or more medical sources, such as medical textbooks, medical societies, medical organizations, medical websites, and the like thereof. For example, and without limitation, target tissue may be received as a function of a medical textbook such as Current Medical Diagnosis and Treatment and/or Diagnostic and Statistical Manual of Mental Disorders, 5^{th} Edition: DSM-5. As a further non-limiting example, target tissue may be received as a function of a medical website such as WebMD.com, and/or MayoClinic.org.

Still referring to FIG. 1, corporeal effect may be identified as a function of target tissue and habit indicator 108 using a corporeal machine-learning model. As used in this disclosure a “corporeal machine-learning model” is a machine-learning model to produce a corporeal effect output given target tissues and habit indicators as inputs; this is in contrast to a non-machine learning software program where the commands to be executed are determined in advance by a user and written in a programming language. Corporeal machine-learning model may include one or more corporeal machine-learning processes such as supervised, unsupervised, or reinforcement machine-learning processes that computing device 104 and/or a remote device may or may not use in the determination of corporeal effect, wherein a remote device is an external device to computing device 104 as described above in detail. A corporeal machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train corporeal machine-learning process as a function of a corporeal training set. As used in this disclosure a “corporeal training set” is a training set that correlates at least target tissue and habit indicator to a corporeal effect. For example, and without limitation, target tissue of cardiac muscle tissue and a habit indicator of consuming excessive concentration of saturated fat may relate to a corporeal effect of increased blood pressure. The corporeal training set may be received as a function of user-entered valuations of target tissues, habit indicators, and/or corporeal effects. Computing device 104 may receive corporeal training set by receiving correlations of target tissues and/or habit indicators that were previously received and/or determined during a previous iteration of determining corporeal effects. The corporeal training set may be received by one or more remote devices that at least correlate a target tissue and habit indicator to a corporeal effect, wherein a remote device is an external device to computing device 104, as described above. Corporeal training set may be received in the form of one or more user-entered correlations of a target tissue and/or habit indicator to a corporeal effect. Additionally or alternatively, a user may include an informed advisor, wherein an informed advisor may include, without limitation nutritionists, lifestyle coaches, family physicians, primary care physicians, internists, pediatricians, pathologists, neurologists, and the like thereof.

Still referring to FIG. 1, computing device 104 may receive corporeal machine-learning model from a remote device that utilizes one or more corporeal machine learning processes, wherein remote device is described above in detail. For example, and without limitation, remote device may include a computing device, external device, processor, and the like thereof. Remote device may perform the corporeal machine-learning process using the corporeal training set to generate corporeal effect and transmit the output to computing device 104. Remote device may transmit a signal, bit, datum, or parameter to computing device 104 that at least relates to corporeal effect. Additionally or alternatively, the remote device may provide an updated machine-learning model. For example, and without limitation, an updated machine-learning model may be comprised of a firmware update, a software update, a corporeal machine-learning process correction, and the like thereof. As a non-limiting example a software update may incorporate a new target tissue that relates to a modified habit indicator. Additionally or alternatively, the updated machine learning model may be transmitted to the remote device, wherein the remote device may replace the corporeal machine-learning model with the updated machine-learning model and determine the physiological as a function of the habit indicator using the updated machine-learning model. The updated machine-learning model may be transmitted by the remote device and received by computing device 104 as a software update, firmware update, or corrected corporeal machine-learning model. For example, and without limitation a corporeal machine-learning model may utilize a neural net machine-learning process, wherein the updated machine-learning model may incorporate polynomial regression machine-learning process. Updated machine learning model may additionally or alternatively include any machine-learning model used as an updated machine learning model as described in U.S. Nonprovisional application Ser. No. 17/106,658, the entirety of which is incorporated herein by reference. In an embodiment, and without limitation, corporeal machine-learning model may identify corporeal effect as a function of one or more classifiers, wherein a classifier is described above in detail.

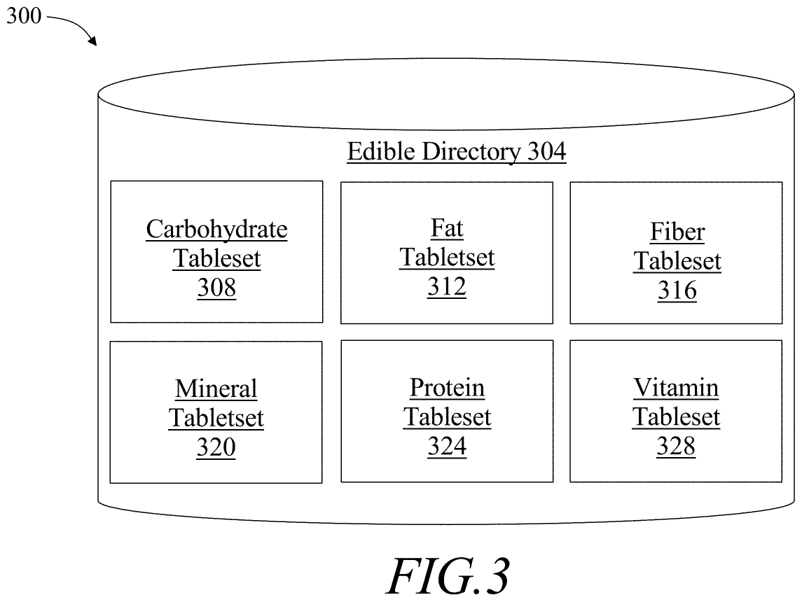

Still referring to FIG. 1, computing device 104 determines an edible 128 as a function of habit profile 112. Still referring to FIG. 1, computing device 104 determines an edible 128 as a function of habit profile 112. As used in this disclosure an “edible” is a source of nourishment that may be consumed by a user such that the user may absorb the nutrients from the source. For example and without limitation, an edible may include legumes, plants, fungi, nuts, seeds, breads, dairy, eggs, meat, cereals, rice, seafood, desserts, dried foods, dumplings, pies, noodles, salads, stews, soups, sauces, sandwiches, and the like thereof. Computing device 104 may determine edible 128 as a function of receiving a nourishment composition. As used in this disclosure a “nourishment composition” is a list and/or compilation of all of the nutrients contained in an edible. As a non-limiting example nourishment composition may include one or more quantities and/or amounts of total fat, including saturated fat and/or trans-fat, cholesterol, sodium, total carbohydrates, including dietary fiber and/or total sugars, protein, vitamin A, vitamin C, thiamin, riboflavin, niacin, pantothenic acid, vitamin b6, folate, biotin, vitamin B12, vitamin D, vitamin E, vitamin K, calcium, iron, phosphorous, iodine, magnesium, zinc, selenium, copper, manganese, chromium, molybdenum, chloride, and the like thereof. In an embodiment, and without limitation, nourishment composition may be obtained as a function of an edible directory, wherein an edible directory is a database of edibles that may be identified as a function of one or more metabolic components as described below in detail, in reference to FIG. 3.

Still referring to FIG. 1, computing device 104 may produce a nourishment desideration as a function of habit profile 112. As used in this disclosure a “nourishment desideration” is requirement and/or necessary amount of nutrients required for a user to consume. As a non-limiting example, nourishment desideration may include a user requirement of 35 g of fiber to be consumed per day. Nourishment desideration may be determined as a function of receiving a nourishment goal. As used in this disclosure a “nourishment goal” is a recommended amount of nutrients that a user should consume. Nourishment goal may be identified by one or more organizations that relate to, represent, and/or study habit dysfunctions in humans, such as the American Medical Association, American College of Lifestyle Medicine, American Red Cross, American Psychological Association, The European Lifestyle Medicine Organization, Lifestyle Medicine Foundation, and the like thereof.

Still referring to FIG. 1, computing device 104 may identify edible 128 as a function of nourishment composition, nourishment desideration, and an edible machine-learning model. As used in this disclosure a “edible machine-learning model” is a machine-learning model to produce an edible output given nourishment compositions and nourishment desiderations as inputs; this is in contrast to a non-machine learning software program where the commands to be executed are determined in advance by a user and written in a programming language. Edible machine-learning model may include one or more edible machine-learning processes such as supervised, unsupervised, or reinforcement machine-learning processes that computing device 104 and/or a remote device may or may not use in the determination of edible 128, wherein a remote device is an external device to computing device 104 as described above in detail. An edible machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train edible machine-learning process as a function of an edible training set. As used in this disclosure an “edible training set” is a training set that correlates at least nourishment composition and nourishment desideration to an edible. For example, and without limitation, nourishment composition of 100 mg of vitamin C and a nourishment desideration of 90 mg of Vitamin C as a function of lung cancer may relate to an edible of oranges. The edible training set may be received as a function of user-entered valuations of nourishment compositions, nourishment desiderations, and/or edibles. Computing device 104 may receive edible training set by receiving correlations of nourishment compositions and/or nourishment desiderations that were previously received and/or determined during a previous iteration of determining edibles. The edible training set may be received by one or more remote devices that at least correlate a nourishment composition and nourishment desideration to an edible, wherein a remote device is an external device to computing device 104, as described above. Edible training set may be received in the form of one or more user-entered correlations of a nourishment composition and/or nourishment desideration to an edible. Additionally or alternatively, a user may include an informed advisor, wherein an informed advisor may include, without limitation nutritionists, lifestyle coaches, family physicians, primary care physicians, internists, pediatricians, pathologists, neurologists, and the like thereof.

Still referring to FIG. 1, computing device 104 may receive edible machine-learning model from a remote device that utilizes one or more edible machine learning processes, wherein remote device is described above in detail. For example, and without limitation, remote device may include a computing device, external device, processor, and the like thereof. Remote device may perform the edible machine-learning process using the edible training set to generate edible 128 and transmit the output to computing device 104. Remote device may transmit a signal, bit, datum, or parameter to computing device 104 that at least relates to edible 128. Additionally or alternatively, the remote device may provide an updated machine-learning model. For example, and without limitation, an updated machine-learning model may be comprised of a firmware update, a software update, an edible machine-learning process correction, and the like thereof. As a non-limiting example a software update may incorporate a new nourishment composition that relates to a modified nourishment desideration. Additionally or alternatively, the updated machine learning model may be transmitted to the remote device, wherein the remote device may replace the edible machine-learning model with the updated machine-learning model and determine the edible as a function of the nourishment desideration using the updated machine-learning model. The updated machine-learning model may be transmitted by the remote device and received by computing device 104 as a software update, firmware update, or corrected edible machine-learning model. For example, and without limitation an edible machine-learning model may utilize a neural net machine-learning process, wherein the updated machine-learning model may incorporate polynomial regression machine-learning process. Updated machine learning model may additionally or alternatively include any machine-learning model used as an updated machine learning model as described in U.S. Nonprovisional application Ser. No. 17/106,658, the entirety of which is incorporated herein by reference. In an embodiment, and without limitation, edible machine-learning model may identify edible 128 as a function of one or more classifiers, wherein a classifier is described above in detail.

Still referring to FIG. 1, computing device 104 may identify edible 128 as a function of a likelihood parameter. As used in this disclosure a “likelihood parameter” is a parameter that identities the probability of a user to consume an edible. As a non-limiting example likelihood parameter may identify a high probability that a user will consume an edible of steaks. As a further non-limiting example likelihood parameter may identify a low probability that a user will consume an edible of beets. Likelihood parameter may be determined as a function of a user taste profile. As used in this disclosure a “user taste profile” is a profile of a user that identifies one or more desires, preferences, wishes, and/or wants that a user has. As a non-limiting example a user taste profile may include a user's preference for cinnamon flavor and/or crunchy textured edibles. Likelihood parameter may be determined as a function of an edible profile. As used in this disclosure an “edible profile” is taste of an edible is the sensation of flavor perceived in the mouth and throat on contact with the edible. Edible profile may include one or more flavor variables. As used in this disclosure a “flavor variable” is a variable associated with the distinctive taste of an edible, wherein a distinctive may include, without limitation sweet, bitter, sour, salty, umami, cool, and/or hot. Edible profile may be determined as a function of receiving flavor variable from a flavor directory. As used in this disclosure a “flavor directory” is a database or other data structure including flavors for an edible. As a non-limiting example flavor directory may include a list and/or collection of edibles that all contain sweet flavor variables. As a further non-limiting example flavor directory may include a list and/or collection of edibles that all contain sour flavor variables. Flavor directory may be implemented similarly to an edible directory as described above in detail. Likelihood parameter may alternatively or additionally include any user taste profile and/or edible profile used as a likelihood parameter as described in U.S. Nonprovisional application Ser. No. 17/032,080, filed on Sep. 25, 2020, and entitled “METHODS, SYSTEMS, AND DEVICES FOR GENERATING A REFRESHMENT INSTRUCTION SET BASED ON INDIVIDUAL PREFERENCES,” the entirety of which is incorporated herein by reference.

Still referring to FIG. 1, computing device 104 generates a nourishment program 132 as a function of edible 128. As used in this disclosure a “nourishment program” is a program consisting of one or more edibles that are to be consumed over a given time period, wherein a time period is a temporal measurement such as seconds, minutes, hours, days, weeks, months, years, and the like thereof. As a non-limiting example nourishment program 132 may consist of recommending broccoli for 8 days. As a further non-limiting example nourishment program 132 may recommend red grapes for a first day, avocado for a second day, and garlic for a third day. Nourishment program 132 may include one or more diet programs such as paleo, keto, vegan, vegetarian, Mediterranean, Dukan, Zone, HCG, and the like thereof. Computing device 104 may develop nourishment program 132 as a function of an intended functional goal. As used in this disclosure an “intended functional goal” is a goal that an edible may generate according to a predicted and/or purposeful plan. As a non-limiting example, intended functional goal may include a treatment goal. As used in this disclosure a “treatment goal” is an intended functional goal that is designed to at least reverse and/or eliminate habit indicator 108, habit profile 112, and/or habit dysfunction. As a non-limiting example, a treatment goal may include reversing the effects of obesity. As a further non-limiting example, a treatment goal includes reversing colitis. Intended functional goal may include a prevention goal. As used in this disclosure a “prevention goal” is an intended functional goal that is designed to at least prevent and/or avert habit indicator 108, habit profile 112, and/or habit dysfunction. As a non-limiting example, a prevention goal may include preventing the development of osteoporosis. Intended functional goal may include a mitigation goal. As used in this disclosure a “mitigation goal” is a functional goal that is designed to reduce the symptoms and/or effects of habit indicator 108, habit profile 112, and/or habit dysfunction. For example, and without limitation, mitigation goal may include reducing the effects of irritable bowl syndrome. Additionally or alternatively, intended functional goal may include one or more goals associated with epigenetic alteration and/or gene therapy to alter a mutation and/or modification associated with a habit dysfunction.

Still referring to FIG. 1, computing device 104 may develop nourishment program 132 as a function of edible 128 and intended functional goal using a nourishment machine-learning model. As used in this disclosure a “nourishment machine-learning model” is a machine-learning model to produce a nourishment program output given edibles and/or intended functional goals as inputs; this is in contrast to a non-machine learning software program where the commands to be executed are determined in advance by a user and written in a programming language. Nourishment machine-learning model may include one or more nourishment machine-learning processes such as supervised, unsupervised, or reinforcement machine-learning processes that computing device 104 and/or a remote device may or may not use in the development of nourishment program 132. Nourishment machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train nourishment machine-learning process as a function of a nourishment training set. As used in this disclosure a “nourishment training set” is a training set that correlates an intended functional goal to an edible. The nourishment training set may be received as a function of user-entered edibles, intended functional goals, and/or nourishment programs. For example, and without limitation, an intended functional goal of treating hypertension may correlate to an edible of pumpkin seeds. Computing device 104 may receive nourishment training by receiving correlations of intended functional goals and/or edibles that were previously received and/or determined during a previous iteration of developing nourishment programs. The nourishment training set may be received by one or more remote devices that at least correlate an intended functional goal and/or edible to a nourishment program, wherein a remote device is an external device to computing device 104, as described above. Nourishment training set may be received in the form of one or more user-entered correlations of an intended functional goal and/or edible to a nourishment program. Additionally or alternatively, a user may include an informed advisor, wherein an informed advisor may include, without limitation nutritionists, lifestyle coaches, family physicians, primary care physicians, internists, pediatricians, and the like thereof.

Still referring to FIG. 1, computing device 104 may receive nourishment machine-learning model from the remote device that utilizes one or more nourishment machine learning processes, wherein a remote device is described above in detail. For example, and without limitation, a remote device may include a computing device, external device, processor, and the like thereof. The remote device may perform the nourishment machine-learning process using the nourishment training set to develop nourishment program 132 and transmit the output to computing device 104. The remote device may transmit a signal, bit, datum, or parameter to computing device 104 that at least relates to nourishment program 132. Additionally or alternatively, the remote device may provide an updated machine-learning model. For example, and without limitation, an updated machine-learning model may be comprised of a firmware update, a software update, a nourishment machine-learning process correction, and the like thereof. As a non-limiting example a software update may incorporate a new intended functional goal that relates to a modified edible. Additionally or alternatively, the updated machine learning model may be transmitted to the remote device, wherein the remote device may replace the nourishment machine-learning model with the updated machine-learning model and develop the nourishment program as a function of the intended functional goal using the updated machine-learning model. The updated machine-learning model may be transmitted by the remote device and received by computing device 104 as a software update, firmware update, or corrected nourishment machine-learning model. For example, and without limitation nourishment machine-learning model may utilize a neural net machine-learning process, wherein the updated machine-learning model may incorporate decision tree machine-learning processes. Updated machine learning model may additionally or alternatively include any machine-learning model used as an updated machine learning model as described in U.S. Nonprovisional application Ser. No. 17/106,658, the entirety of which is incorporated herein by reference.

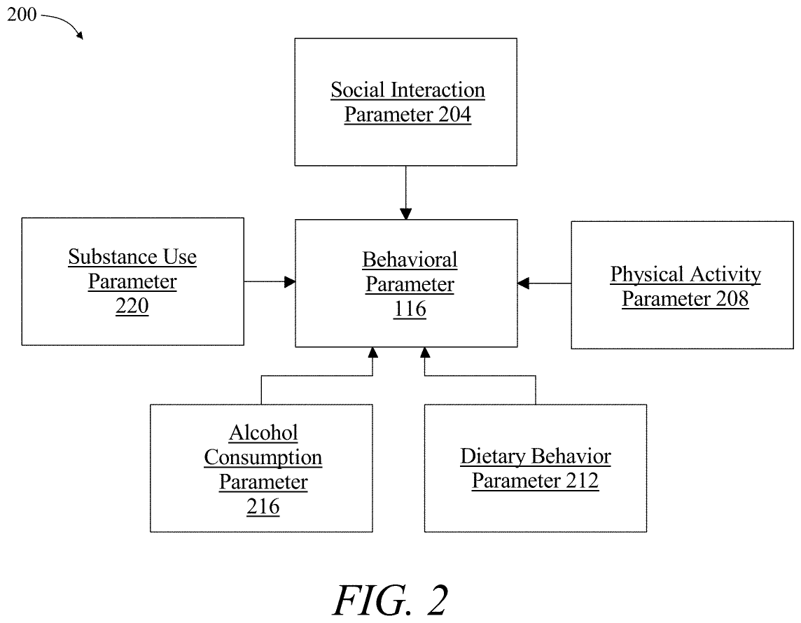

Now referring to FIG. 2, an exemplary embodiment 200 of a behavioral parameter 116 is illustrated. Behavioral parameter may include a social interaction parameter 204. As used in this disclosure a “social interaction parameter” is a measurable value representing a magnitude interactions an individual has with another individual in a given time period, wherein a time period is described above in detail. For example and without limitation, social interaction parameter 204 may denote that an individual has 15 social interactions per day. As a further non-limiting example, social interaction parameter 204 may denote that an individual has 200 social interactions per day. As a further non-limiting example, social interaction parameter 204 may denote that an individual has one or more social relations, regulated interactions, regular interactions, repeated interactions, social contacts, and the like thereof with other individuals in a time period such as seconds, minutes, hours, days, months, weeks, years, and the like thereof. In an embodiment, and without limitation, behavioral parameter 116 may include a physical activity parameter 208. As used in this disclosure a “physical activity parameter” is a measurable value representing a magnitude of physical activity that an individual performs in a given time period, wherein a time period is described above in detail. For example, and without limitation, physical activity parameter 208 may denote that an individual performs 25 minutes of physical activity in a day. As a further non-limiting example, physical activity parameter 208 may denote that an individual performs a physical activity of golfing for 4 hours a week. As a further non-limiting example, physical activity parameter 208 may denote that an individual performs a physical activity of swimming for 30 minutes a month.

In an embodiment, and still referring to FIG. 2, behavioral parameter 116 may include a dietary behavior parameter 212. As used in this disclosure a “dietary behavior parameter” is a parameter denoting an edible consumption habit of an individual in a given time period, wherein a time period is described above in detail. For example, and without limitation, dietary behavior parameter 212 may denote that an individual regularly consumes chips and/or cookies throughout a day. As a further non-limiting example, dietary behavior parameter 212 may denote that an individual consumes a fish and/or seafood three times week. As a further non-limiting example, dietary behavior parameter 212 may denote that an individual adheres to one or more diet plans and/or programs, such as keto, vegan, whole foods, paleo, vegetarian, pescatarian, kosher, and the like thereof. In an embodiment, and without limitation, behavioral parameter 116 may include an alcohol consumption parameter 216. As used in this disclosure an “alcohol consumption parameter” is a measurable value representing a magnitude of alcoholic beverages that an individual consumes in a given time period, wherein a time period is described above in detail. For example, and without limitation, alcohol consumption parameter 216 may denote that an individual consumes 5 alcoholic beverages daily. As a further non-limiting example, alcohol consumption parameter 216 may denote that an individual consumes 20 alcoholic beverages over the weekend. In an embodiment, and without limitation, behavioral parameter 116 may include a substance use parameter 220. As used in this disclosure a “substance use parameter” is a parameter denoting the use and/or abuse of illicit drugs and/or chemicals by an individual. For example, and without limitation, substance use parameter 220 may denote that individual consumes and/or ingests methamphetamine. As a further non-limiting example, substance use parameter 220 may denote that the individual abuses fentanyl and/or morphine.

Now referring to FIG. 3, an exemplary embodiment 300 of an edible directory 304 according to an embodiment of the invention is illustrated. Edible directory 304 may be implemented, without limitation, as a relational databank, a key-value retrieval databank such as a NOSQL databank, or any other format or structure for use as a databank that a person skilled in the art would recognize as suitable upon review of the entirety of this disclosure. Edible directory 304 may alternatively or additionally be implemented using a distributed data storage protocol and/or data structure, such as a distributed hash table or the like. Edible directory 304 may include a plurality of data entries and/or records as described above. Data entries in a databank may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a databank may store, retrieve, organize, and/or reflect data and/or records as used herein, as well as categories and/or populations of data consistently with this disclosure. Edible directory 304 may include a carbohydrate tableset 308. Carbohydrate tableset 308 may relate to a nourishment composition of an edible with respect to the quantity and/or type of carbohydrates in the edible. As a non-limiting example, carbohydrate tableset 308 may include monosaccharides, disaccharides, oligosaccharides, polysaccharides, and the like thereof. Edible directory 304 may include a fat tableset 312. Fat tableset 312 may relate to a nourishment composition of an edible with respect to the quantity and/or type of esterified fatty acids in the edible. Fat tableset 312 may include, without limitation, triglycerides, monoglycerides, diglycerides, phospholipids, sterols, waxes, and free fatty acids. Edible directory 304 may include a fiber tableset 316. Fiber tableset 316 may relate to a nourishment composition of an edible with respect to the quantity and/or type of fiber in the edible. As a non-limiting example, fiber tableset 316 may include soluble fiber, such as beta-glucans, raw guar gum, psyllium, inulin, and the like thereof as well as insoluble fiber, such as wheat bran, cellulose, lignin, and the like thereof. Edible directory 304 may include a mineral tableset 320. Mineral tableset 320 may relate to a nourishment composition of an edible with respect to the quantity and/or type of minerals in the edible. As a non-limiting example, mineral tableset 320 may include calcium, phosphorous, magnesium, sodium, potassium, chloride, sulfur, iron, manganese, copper, iodine, zing, cobalt, fluoride, selenium, and the like thereof. Edible directory 304 may include a protein tableset 324. Protein tableset 324 may relate to a nourishment composition of an edible with respect to the quantity and/or type of proteins in the edible. As a non-limiting example, protein tableset 324 may include amino acids combinations, wherein amino acids may include, without limitation, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, and the like thereof. Edible directory 304 may include a vitamin tableset 328. Vitamin tableset 328 may relate to a nourishment composition of an edible with respect to the quantity and/or type of vitamins in the edible. As a non-limiting example, vitamin tableset 328 may include vitamin A, vitamin B₁, vitamin B₂, vitamin B₃, vitamin B₅, vitamin B₆, vitamin B₇, vitamin B₉, vitamin B₁₂, vitamin C, vitamin D, vitamin E, vitamin K, and the like thereof.

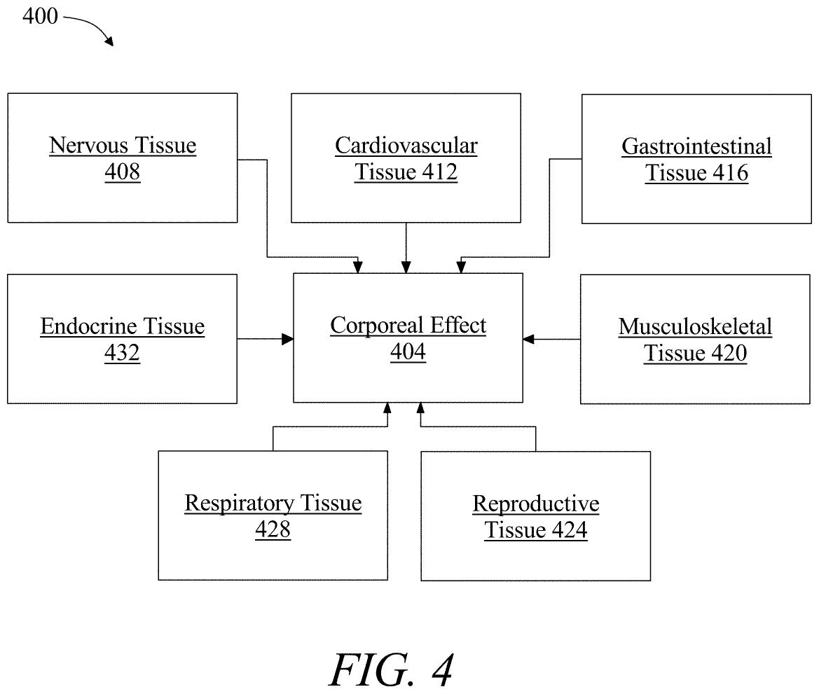

Now referring to FIG. 4, an exemplary embodiment 400 of a corporeal effect 404 is illustrated. As used in this disclosure a “corporeal effect” effects and/or impacts that a habit has on an individual's health system. For example, and without limitation, corporeal effect may include one or more effects on the cells, tissues, organs, and the like thereof of the human body. In an embodiment, corporeal effect 404 may include an effect on a nervous tissue 408. As used in this disclosure “nervous tissue” is a cell and/or group of cells that are associated with the transmission of electrical and/or chemical signals in the human body. For example, and without limitation, nervous tissue 408 may include one or more tissues such as the brain, spinal cord, neurons, neuroglia, and the like thereof. In an embodiment, corporeal effect 404 may include an effect on a cardiovascular tissue 412. As used in this disclosure “cardiovascular tissue” is a cell and/or group of cells that are associated with the circulation of blood in the body. For example, and without limitation, cardiovascular tissue 412 may include one or more tissues such as the heart, cardiac muscle, specialized conductive tissue, valves, blood vessels, connective tissue, and the like thereof. In an embodiment, corporeal effect 404 may include an effect on a gastrointestinal tissue 416. As used in this disclosure “gastrointestinal tissue” is a cell and/or group of cells that are associated with the ingestion, digestion, and/or absorption of nutrients. For example, and without limitation, gastrointestinal tissue 416 may include one or more tissues such as the esophagus, stomach, bowel, mucosa, submucosa, muscular tissue, serosa, adventitia, and the like thereof.

Still referring to FIG. 4, corporeal effect 404 may include an effect on a musculoskeletal tissue 420. As used in this disclosure “musculoskeletal tissue” is a cell and/or group of cells that are associated with the support of the body, motion of the body, and/or protection of vital organs. For example, and without limitation, musculoskeletal tissue 420 may include one or more tissues such as bones, muscles, cartilage, tendons, ligaments, joints, connective tissue, and the like thereof. In an embodiment, corporeal effect 404 may include an effect on a reproductive tissue 424. As used in this disclosure “reproductive tissue” is a cell and/or group of cells that are associated with procreation. For example, and without limitation, reproduction tissue 424 may include one or more tissues such as the testes, seminal vesicles, prostate gland, bulbourethral gland, ovaries, uterus, oviducts, and the like thereof. In an embodiment, corporeal effect 404 may include an effect on a respiratory tissue 428. As used in this disclosure “respiratory tissue” is a cell and/or group of cells that are associated with the external exchange of gases. For example, and without limitation, respiratory tissue 428 may include one or more tissues such as the mouth, nose, sinuses, pharynx, trachea, bronchial tubes, lungs, diaphragm, alveoli, bronchioles, lung lobes, pleura, cilia, epiglottis, larynx, and the like thereof. In an embodiment, corporeal effect 404 may include an effect on a endocrine tissue 432. As used in this disclosure “endocrine tissue” is a cell and/or group of cells that are associated with the control of mood, growth, metabolism, and/or reproduction. For example, and without limitation, endocrine tissue 432 may include one or more tissues such as the hypothalamus, pituitary gland, thyroid gland, parathyroid gland, adrenal gland, pineal body, ovaries, testes, and the like thereof.

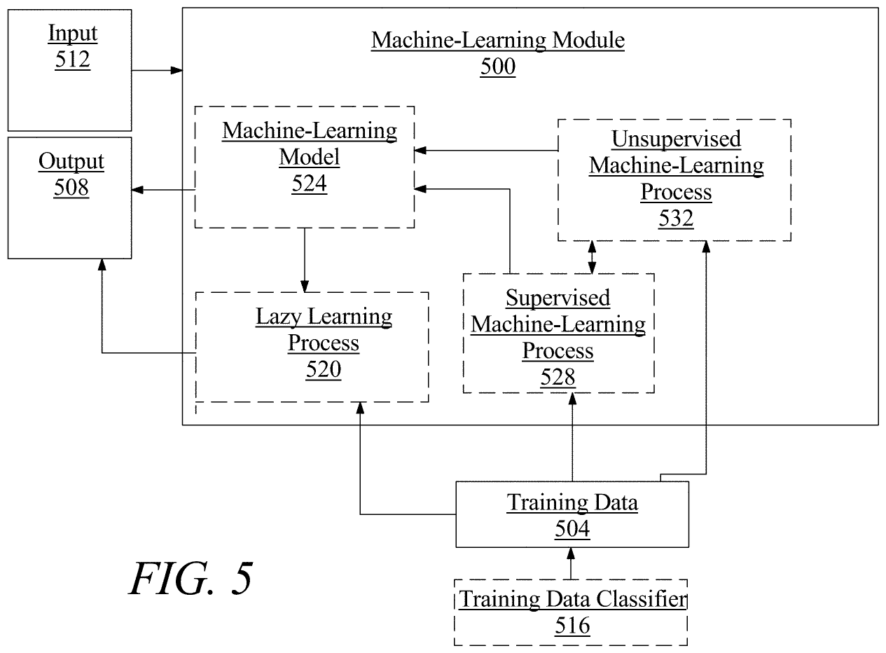

Referring now to FIG. 5, an exemplary embodiment of a machine-learning module 500 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 504 to generate an algorithm that will be performed by a computing device/module to produce outputs 508 given data provided as inputs 512; this is in contrast to a non-machine learning software program where the commands to be executed are determined in advance by a user and written in a programming language.

Still referring to FIG. 5, “training data,” as used herein, is data containing correlations that a machine-learning process may use to model relationships between two or more categories of data elements. For instance, and without limitation, training data 504 may include a plurality of data entries, each entry representing a set of data elements that were recorded, received, and/or generated together; data elements may be correlated by shared existence in a given data entry, by proximity in a given data entry, or the like. Multiple data entries in training data 504 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 504 according to various correlations; correlations may indicate causative and/or predictive links between categories of data elements, which may be modeled as relationships such as mathematical relationships by machine-learning processes as described in further detail below. Training data 504 may be formatted and/or organized by categories of data elements, for instance by associating data elements with one or more descriptors corresponding to categories of data elements. As a non-limiting example, training data 504 may include data entered in standardized forms by persons or processes, such that entry of a given data element in a given field in a form may be mapped to one or more descriptors of categories. Elements in training data 504 may be linked to descriptors of categories by tags, tokens, or other data elements; for instance, and without limitation, training data 504 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 5, training data 504 may include one or more elements that are not categorized; that is, training data 504 may not be formatted or contain descriptors for some elements of data. Machine-learning algorithms and/or other processes may sort training data 504 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 504 to be made applicable for two or more distinct machine-learning algorithms as described in further detail below. Training data 504 used by machine-learning module 500 may correlate any input data as described in this disclosure to any output data as described in this disclosure. As a non-limiting illustrative example inputs of behavioral divergences and/or habit indicators may relate to an output of a habit profile.

Further referring to FIG. 5, training data may be filtered, sorted, and/or selected using one or more supervised and/or unsupervised machine-learning processes and/or models as described in further detail below; such models may include without limitation a training data classifier 516. Training data classifier 516 may include a “classifier,” which as used in this disclosure is a machine-learning model as defined below, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 500 may generate a classifier using a classification algorithm, defined as a processes whereby a computing device and/or any module and/or component operating thereon derives a classifier from training data 504. Classification may be performed using, without limitation, linear classifiers such as without limitation logistic regression and/or naïBayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 516 may classify elements of training data to sub-categories of behavioral divergences such as low, medium, and/or high divergences that denote the distance from the behavioral normality.

Still referring to FIG. 5, machine-learning module 500 may be configured to perform a lazy-learning process 520 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 504. Heuristic may include selecting some number of highest-ranking associations and/or training data 504 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 5, machine-learning processes as described in this disclosure may be used to generate machine-learning models 524. A “machine-learning model,” as used in this disclosure, is a mathematical and/or algorithmic representation of a relationship between inputs and outputs, as generated using any machine-learning process including without limitation any process as described above, and stored in memory; an input is submitted to a machine-learning model 524 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 524 may be generated by creating an artificial neural network, such as a convolutional neural network comprising an input layer of nodes, one or more intermediate layers, and an output layer of nodes. Connections between nodes may be created via the process of “training” the network, in which elements from a training data 504 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 5, machine-learning algorithms may include at least a supervised machine-learning process 528. At least a supervised machine-learning process 528, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include behavioral divergences and/or habit indicators as described above as inputs, habit profiles as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 504. Persons skilled in the art, upon reviewing the entirety of this disclosure, will be aware of various possible variations of at least a supervised machine-learning process 528 that may be used to determine relation between inputs and outputs. Supervised machine-learning processes may include classification algorithms as defined above.

Further referring to FIG. 5, machine learning processes may include at least an unsupervised machine-learning processes 532. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 5, machine-learning module 500 may be designed and configured to create a machine-learning model 524 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 5, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

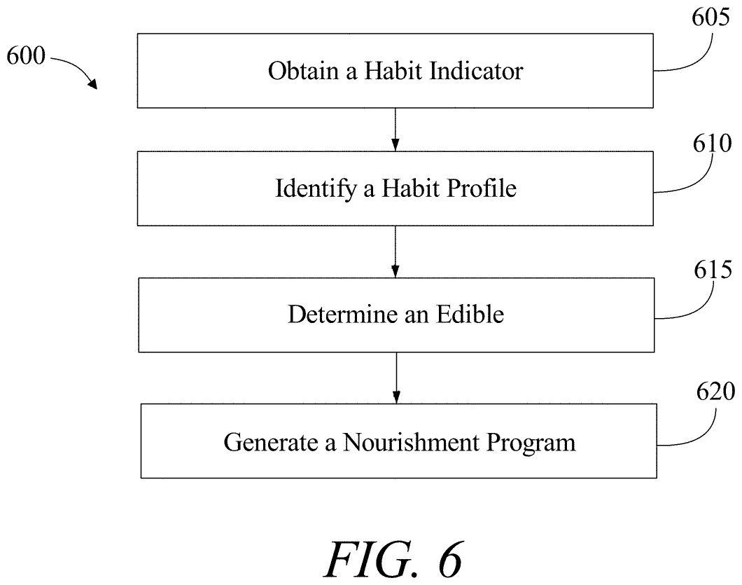

Now referring to FIG. 6, an exemplary embodiment 600 of a method for generating a habit dysfunction nourishment program is illustrated. At step 605, a computing device 104 obtains a habit indicator 108. Computing device 104 includes any of the computing device 104 as described above, in reference to FIGS. 1-5. Habit indicator 108 includes any of the habit indicator 108 as described above, in reference to FIGS. 1-5.

Still referring to FIG. 6, at step 610, computing device 104 identifies a habit profile 112. Habit profile 112 includes any of the habit profile 112 as described above, in reference to FIGS. 1-5. Computing device 104 identifies habit profile 112 as a function of retrieving a behavioral parameter 116. Behavioral parameter 116 includes any of the behavioral parameter 116 as described above, in reference to FIGS. 1-5. Computing device 104 identifies habit profile 112 by determining a behavioral divergence 120. Behavioral divergence 120 includes any of the behavioral divergence 120 as described above, in reference to FIGS. 1-5. Computing device 104 identifies habit profile 112 as a function of behavioral parameter 116 and habit indicator 108 using a habit machine-learning model 124. Habit machine-learning model 124 includes any of the habit machine-learning model 124 as described above, in reference to FIGS. 1-5.

Still referring to FIG. 6, at step 615, computing device 104 determines an edible 128 as a function of habit profile 112. Edible 128 includes any of the edible 128 as described above, in reference to FIGS. 1-5.

Still referring to FIG. 6, at step 620, computing device 104 generates a nourishment program 132 as a function of edible 128. Nourishment program 132 includes any of the nourishment program 132 as described above, in reference to FIGS. 1-5.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

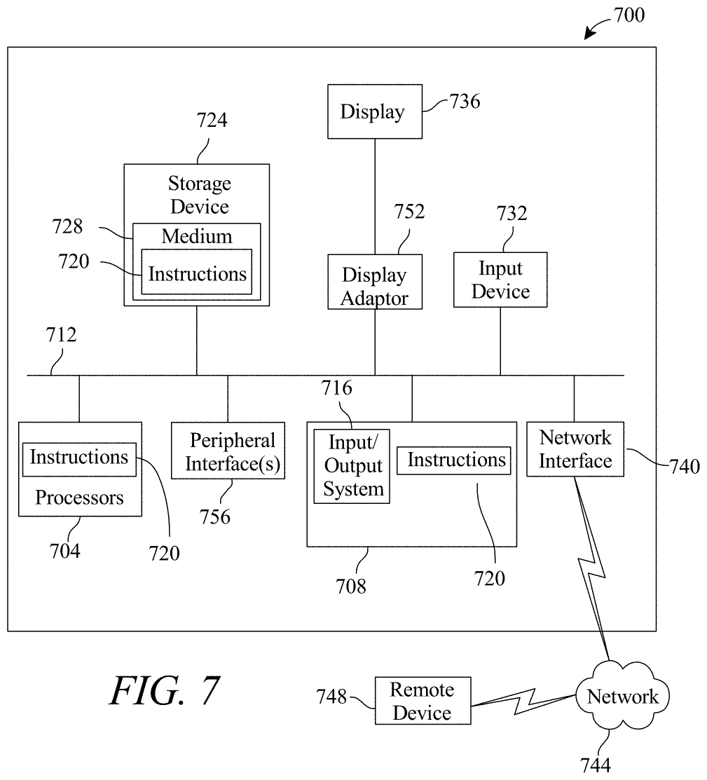

FIG. 7 shows a diagrammatic representation of one embodiment of a computing device in the exemplary form of a computer system 700 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 700 includes a processor 704 and a memory 708 that communicate with each other, and with other components, via a bus 712. Bus 712 may include any of several types of bus structures including, but not limited to, a memory bus, a memory controller, a peripheral bus, a local bus, and any combinations thereof, using any of a variety of bus architectures.

Processor 704 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 704 may be organized according to Von Neumann and/or Harvard architecture as a non-limiting example. Processor 704 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 708 may include various components (e.g., machine-readable media) including, but not limited to, a random-access memory component, a read only component, and any combinations thereof. In one example, a basic input/output system 716 (BIOS), including basic routines that help to transfer information between elements within computer system 700, such as during start-up, may be stored in memory 708. Memory 708 may also include (e.g., stored on one or more machine-readable media) instructions (e.g., software) 720 embodying any one or more of the aspects and/or methodologies of the present disclosure. In another example, memory 708 may further include any number of program modules including, but not limited to, an operating system, one or more application programs, other program modules, program data, and any combinations thereof.

Computer system 700 may also include a storage device 724. Examples of a storage device (e.g., storage device 724) include, but are not limited to, a hard disk drive, a magnetic disk drive, an optical disc drive in combination with an optical medium, a solid-state memory device, and any combinations thereof. Storage device 724 may be connected to bus 712 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 724 (or one or more components thereof) may be removably interfaced with computer system 700 (e.g., via an external port connector (not shown)). Particularly, storage device 724 and an associated machine-readable medium 728 may provide nonvolatile and/or volatile storage of machine-readable instructions, data structures, program modules, and/or other data for computer system 700. In one example, software 720 may reside, completely or partially, within machine-readable medium 728. In another example, software 720 may reside, completely or partially, within processor 704.

Computer system 700 may also include an input device 732. In one example, a user of computer system 700 may enter commands and/or other information into computer system 700 via input device 732. Examples of an input device 732 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 732 may be interfaced to bus 712 via any of a variety of interfaces (not shown) including, but not limited to, a serial interface, a parallel interface, a game port, a USB interface, a FIREWIRE interface, a direct interface to bus 712, and any combinations thereof. Input device 732 may include a touch screen interface that may be a part of or separate from display 736, discussed further below. Input device 732 may be utilized as a user selection device for selecting one or more graphical representations in a graphical interface as described above.

A user may also input commands and/or other information to computer system 700 via storage device 724 (e.g., a removable disk drive, a flash drive, etc.) and/or network interface device 740. A network interface device, such as network interface device 740, may be utilized for connecting computer system 700 to one or more of a variety of networks, such as network 744, and one or more remote devices 748 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 744, may employ a wired and/or a wireless mode of communication. In general, any network topology may be used. Information (e.g., data, software 720, etc.) may be communicated to and/or from computer system 700 via network interface device 740.

Computer system 700 may further include a video display adapter 752 for communicating a displayable image to a display device, such as display device 736. Examples of a display device include, but are not limited to, a liquid crystal display (LCD), a cathode ray tube (CRT), a plasma display, a light emitting diode (LED) display, and any combinations thereof. Display adapter 752 and display device 736 may be utilized in combination with processor 704 to provide graphical representations of aspects of the present disclosure. In addition to a display device, computer system 700 may include one or more other peripheral output devices including, but not limited to, an audio speaker, a printer, and any combinations thereof. Such peripheral output devices may be connected to bus 712 via a peripheral interface 756. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve systems and methods according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A system for generating a habit dysfunction nourishment program, the system comprising:
a computing device, the computing device configured to:
obtain a habit indicator;
identify a habit profile, wherein identifying the habit profile further comprises:
retrieving a behavioral parameter;
determining a behavioral divergence as a function of the behavioral parameter; and
identifying the habit profile as a function of the behavioral divergence and the habit indicator using a habit machine-learning model;
determine an edible as a function of the habit profile; and
generate a nourishment program as a function of the edible.

2. The system of claim 1, wherein obtaining the habit indicator further comprises receiving a habit input as a function of a monitoring device and obtaining the habit indicator as a function of the habit input.

3. The system of claim 1, wherein the habit indicator includes a biomarker.

4. The system of claim 1, wherein retrieving the behavioral parameter further comprises obtaining a geolocation element and retrieving the behavioral parameter as a function of the geolocation element.

5. The system of claim 4, wherein the geolocation element includes an industrialization vector.

6. The system of claim 1, wherein determining the behavioral divergence further comprises:
obtaining a behavior normality; and
determining the behavioral divergence as a function of the behavior normality and a divergence threshold.

7. The system of claim 1, wherein identifying the habit profile further comprises determining a habit dysfunction and producing the habit profile as a function of the habit dysfunction.

8. The system of claim 7, wherein the habit dysfunction includes a non-communicable ailment.

9. The system of claim 1, wherein identifying the habit profile further comprises determining a temporal element and identifying the habit profile as a function of the temporal element.

10. The system of claim 1, wherein identifying the habit profile further comprises determining a corporeal effect and identifying the habit profile as a function of the corporeal effect.

11. A method for generating a habit dysfunction nourishment program, the method comprising:
obtaining, by a computing device, a habit indicator;
identifying, by the computing device, a habit profile, wherein identifying the habit profile further comprises:
retrieving a behavioral parameter;
determining a behavioral divergence as a function of the behavioral parameter; and
identifying the habit profile as a function of the behavioral divergence and the habit indicator using a habit machine-learning model;
determining, by the computing device, an edible as a function of the habit profile; and
generating, by the computing device, a nourishment program as a function of the edible.

12. The method of claim 11, wherein obtaining the habit indicator further comprises receiving a habit input as a function of a monitoring device and obtaining the habit indicator as a function of the habit input.

13. The method of claim 11, wherein the habit indicator includes a biomarker.

14. The method of claim 11, wherein retrieving the behavioral parameter further comprises obtaining a geolocation element and retrieving the behavioral parameter as a function of the geolocation element.

15. The method of claim 14, wherein the geolocation element includes an industrialization vector.

16. The method of claim 11, wherein determining the behavioral divergence further comprises:
obtaining a behavior normality; and
determining the behavioral divergence as a function of the behavior normality and a divergence threshold.

17. The method of claim 11, wherein identifying the habit profile further comprises determining a habit dysfunction and producing the habit profile as a function of the habit dysfunction.

18. The method of claim 17, wherein the habit dysfunction includes a non-communicable ailment.

19. The method of claim 11, wherein identifying the habit profile further comprises determining a temporal element and identifying the habit profile as a function of the temporal element.

20. The method of claim 11, wherein identifying the habit profile further comprises determining a corporeal effect and identifying the habit profile as a function of the corporeal effect.
